(19) United States
(12) Patent Application Publication
(10) Pub. No.: US 2026/0216464 A1
(43) Pub. Date: Jul. 30, 2026

(54) METHOD AND DEVICE FOR DETERMINING SUBSTANCE PROPORTIONS OF A FLUID MIXTURE OF SUBSTANCES AND MEDICAL DEVICE

(71) Applicants: WEINMANN EMERGENCY MEDICAL TECHNOLOGY GMBH + CO. KG, Hamburg (DE); CORSCIENCE GMBH & CO. KG, Erlangen (DE)

(72) Inventors: Frank HERRMANN, Barmstedt (DE); Barbara TRABOLD, Uttenreuth (DE); Annika POLERT, Norderstedt (DE)

(21) Appl. No.: 19/151,705

(22) PCT Filed: Nov. 9, 2023
(86) PCT No.: PCT/DE2023/100849

§ 371 Date: Jul. 29, 2025

(30) Foreign Application Priority Data
Feb. 1, 2023 (DE) 10 2023 102 394.2

(51) Int. Cl. A61M 16/10

(52) U.S. Cl. CPC A61M 16/1005; A61M 2016/1025; A61M 2016/103; A61M 2205/3303; A61M 2205/3334

(57) ABSTRACT

A method and a device for determining substance proportions of a fluid mixture of substances and to a medical device. At least two different non-selective measuring devices or measuring methods are used for measuring independent substance properties of a mixture of substances in order to determine the proportions of substances in the mixture of substances by solving a system of equations.

The invention relates to a method for determining substance proportions of a fluid mixture of substances, in particular of gases or liquids, using at least two sensors.

In addition, the invention relates to a device for determining substance proportions of a fluid mixture of substances, in particular of gases or liquids, the device having at least two sensors for this purpose.

Furthermore, the invention relates to a medical device comprising such a device for determining substance proportions of a fluid mixture of substances.

To analyze the composition of fluid (liquid or gaseous) mixtures of substances, measurement methods are usually used which largely use selective properties to determine individual components of the mixture of substances, such as spectroscopic or chemical measurement technologies. However, such selective measurement methods are often complex and/or slow. A high-resolution, selective analysis of the complete composition of a mixture of substances is therefore usually not possible. In some applications, even the selective determination of a single desired substance proportion of a mixture of substances is problematic.

Measurements of the composition of mixtures of substances by capturing several measurement signals in relation to the mixture of substances are state of the art. However, a single substance property is usually evaluated several times by varying an accompanying parameter and examined for component-specific characteristics. For example, the light absorption of a gas/gas mixture is analyzed in an absorption measurement. The measuring wavelength is changed, and the absorption is measured at different wavelengths. By scanning over a wavelength range, the absorption behavior at certain wavelengths can be used to draw conclusions about several gas concentrations or even the complete gas composition. There are many similar methods which, as in this example, analyze a specific substance property (e.g. absorption) using a specific measurement method (e.g. absorption spectroscopy) using a varying parameter (e.g. wavelength) and thus measure the composition of a gas mixture.

These methods are often very complex and expensive, and the equipment required to implement them is large. Mobile use of such devices, for example in a moving emergency vehicle with high shock and vibration loads, is also often not possible.

Such methods and devices are also used in medical technology, for example in respiratory technology, where the composition of the respiratory gas in particular is determined. The rapid selective measurement of oxygen (O2), for example, currently requires complex, expensive measurement methods that are also difficult to integrate into compact devices. In contrast to CO2 measurement, where a filter is placed in front of the sensor on the receiver side, for example, which only allows a CO2-relevant wavelength, this is not possible with O2 measurement. There is no corresponding characteristic absorption range for O2, meaning that optical O2 measurement can only be realized with very finely tunable lasers. However, this procedure is very expensive and time-consuming. The breath-resolved measurement of the oxygen concentration is important for good patient care but has so far been difficult to implement for compact devices. The solution approach described here is intended to show that a combination of primarily non-selective measurement methods allows the gas composition to be determined and thus the oxygen concentration can still be determined.

One object of the invention is to create a device for determining substance proportions of a fluid mixture of substances which at least partially eliminates the disadvantages of the prior art described above.

According to the invention, this object is achieved by a device for determining substance proportions of a fluid mixture of substances according to patent claim 1.

A further object of the invention is to create a medical device with which the above-described disadvantages of the prior art in determining the substance proportions of a fluid mixture of substances are at least partially eliminated.

According to the invention, this object is achieved by a medical device according to patent claim 11.

In addition, it is an object of the invention to provide a method for determining substance proportions of a fluid mixture of substances which at least partially eliminates the disadvantages of the prior art described above.

According to the invention, this object is achieved by a method according to patent claim 13.

Advantageous embodiments of the invention are claimed in the dependent patent claims.

The following disclosed features of a device for determining substance proportions of a fluid mixture of substances, of a medical device and of a method for determining substance proportions of a fluid mixture of substances are part of the invention both individually and in all practicable combinations.

The basic idea of the invention is the combination of several non-selective measuring methods or measuring devices for determining substance proportions of a fluid mixture of substances. Due to the higher speed of non-selective measuring methods, overall faster measurements can be carried out to determine at least one substance proportion of a mixture of substances.

Non-selective measuring methods or measuring devices are such methods or devices with which a certain substance proportion of a mixture of substances cannot be determined directly or with the required accuracy or robustness and exclusively on the basis of the measurement data obtained with this method or device, as it is possible with selective measuring methods or measuring devices.

In embodiments of the invention, however, scanning is not primarily performed over a single substance property, but at least two different and independent substance properties are measured and evaluated.

Independent substance properties are those properties of the respective substance or mixture of substances that do not influence each other. These include, for example, the thermal conductivity, the heat capacity, the speed of sound in the respective substance or mixture of substances, the molar mass, the dynamic viscosity, the permittivity and the refractive index or the speed of light in the respective substance or mixture of substances, which can be used with the known measuring methods for the non-selective determination of the substance proportions of a mixture of substances.

For example, in embodiments of the invention, the following sensors can be used to measure these substance properties.

The thermal conductivity and heat capacity are measured, for example, using a sensor comprising at least one heating element and a temperature sensor. If the fluid mixture is at rest, the temperature measurement at a known heater temperature can be used to infer the thermal conductivity of the fluid mixture after calibrating the sensor. If the temperature of the heater is modulated, the delay with which a temperature change can be detected at the temperature sensor is a measure of the heat capacity of the fluid mixture of substances. In principle, thermal conductivity and heat capacity can therefore be measured with a suitable sensor device.

In embodiments of the invention, the device for determining substance proportions of a fluid mixture of substances has at least one thermal conductivity sensor and/or heat capacity sensor. In embodiments of the invention, this is designed as an integrated sensor chip.

In embodiments of the invention, the speed of sound is measured using ultrasonic technology. Corresponding sensors are already available on the market.

In embodiments of the invention, the device for determining substance proportions of a fluid mixture of substances has at least one speed of sound sensor based on ultrasonic technology.

The molar mass can be derived from the speed of sound. The measurement using other methods is very time-consuming.

In embodiments of the invention, the device for determining substance proportions of a fluid mixture of substances has at least one sensor for determining the molar mass based on the determination of the speed of sound.

The dynamic viscosity can be measured, for example, via the pressure drop in, for example, a thin tube in which a laminar flow has been set. The pressure drop is directly proportional to the dynamic viscosity.

In embodiments of the invention, the device for determining substance proportions of a fluid mixture of substances has at least one sensor for determining the dynamic viscosity based on the determination of a pressure drop.

The permittivity of a mixture of substances can be measured using a plate capacitor through which the fluid mixture of substances flows. The change in capacitance is also directly proportional to the permittivity. Among the gases to be measured in ventilation technology, water vapor is particularly prominent, since water vapor differs from the other relevant gases by more than a power of ten.

In embodiments of the invention, the device for determining substance proportions of a fluid mixture of substances has at least one permittivity sensor.

The refractive index or the speed of light of different gases differs very little, so that in corresponding embodiments of the invention interferometers are used as refractive index sensor or speed of light sensor.

In embodiments of the invention, the device for determining substance proportions of a fluid mixture of substances has at least one refractive index sensor and/or a light velocity sensor comprising at least one interferometer.

If the composition of a mixture of substances with a number of X different substances is to be determined completely, at least X−1 independent non-selective measurements of different substance properties are required in order to be able to solve the resulting system of equations completely and unambiguously.

In embodiments of the invention, use is made of the fact that in many applications only certain substance proportions of a mixture of substances are of interest and/or that certain substances do not influence the substance properties of the mixture of substances to be measured, or only to a small extent.

This allows to reduce the required number of at least necessary non-selective measuring methods/measuring devices of independent substance properties to a value less than or equal to X−1, where X is the number of relevant substance proportions.

A device according to the invention for determining substance proportions of a fluid mixture of substances is designed to determine the proportion of at least one substance in a fluid mixture of substances.

A device according to the invention for determining substance proportions of a fluid mixture of substances has at least two different non-selective measuring devices, each of which measures a substance property of the mixture of substances, the substance properties measured with the measuring devices being different and independent of one another.

Furthermore, a device according to the invention for determining substance proportions of a fluid mixture of substances has at least one evaluation unit for evaluating the measured values of the at least two different substance properties measured by the measuring devices and for determining at least one substance proportion in the mixture of substances from the combination of the measured substance properties.

In advantageous embodiments of the invention with a number X of proportions to be determined in the fluid mixture of substances, the device for determining substance proportions of a fluid mixture of substances has at least X−1 non-selective measuring devices with which various independent substance properties of the mixture of substances can be measured.

In embodiments of the invention, at least two of the non-selective measuring devices are designed to carry out the respective measurements simultaneously or at least partially overlapping in time.

In embodiments of the invention, all non-selective measuring devices are designed to carry out the respective measurements simultaneously or at least partially overlapping in time.

If the various sensors are arranged one behind the other in the volume flow of the fluid mixture of substances, a time offset of the measured values occurs, since a specific volume reaches the individual sensors one after the other. If the volume flow of the fluid mixture of substances is known, the time offset can be compensated for in corresponding embodiments of the invention, so that the measured values of all sensors can be synchronized. For this purpose, in preferred embodiments, the device for determining the substance proportions of a fluid mixture of substances has a volume flow sensor.

In embodiments of the invention, the device for determining substance proportions of a fluid mixture of substances is designed to determine the proportion of O2 and/or CO2 in the mixture of substances.

In embodiments of the invention, at least one measuring device of the device for determining substance proportions of a fluid mixture of substances has at least one sensor for determining one of the substance properties thermal conductivity, heat capacity, speed of sound (e.g. ultrasonic sensor), molar mass, dynamic viscosity, permittivity or refractive index or speed of light.

In embodiments of the invention, the device for determining substance proportions of a fluid mixture of substances additionally has at least one selective sensor for selectively determining at least one substance proportion in the mixture of substances.

In embodiments of the invention, the at least one selective sensor is designed for NDIR absorption measurement for determining the CO2 content, as a lambda probe for determining the O2 content, as a paramagnetic sensor for determining the O2 content, as an optical sensor such as an optrode for measurement by means of fluorescence quenching (in particular a pO2 optrode), as an electrochemical gas sensor or as a humidity sensor (in particular by means of permittivity measurement).

In embodiments of the invention, it is known which substances the mixture of substances contains or which substances the mixture of substances contains that influence the properties of the mixture of substances to a relevant extent.

In embodiments of the invention, the device for determining substance proportions of a fluid mixture of substances is designed for the iterative determination of at least one substance property. This allows the measurement accuracy to be improved in corresponding embodiments.

In embodiments of the invention, the device for determining substance proportions of a fluid mixture of substances has more than X−1 non-selective measuring devices. This allows to increase the robustness and/or the accuracy of the measurement. Furthermore, in embodiments of the invention, a plausibility check and/or a check of the mixture of substances for unknown substances with a relevant influence on the measurement(s) is thereby implemented.

In embodiments of the invention, the device for determining substance proportions of a fluid mixture of substances has an evaluation unit with which the substance proportion of at least one substance in the mixture of substances can be determined by setting up a system of equations from the measured substance properties and the solution of the system of equations.

In embodiments of the invention, the system of equations to be solved can be linear or non-linear.

The system of equations to be set up comprises the measured substance properties and their mathematical dependencies on the individual substances of the mixture of substances, whereby at least the relevant components of the mixture of substances are taken into account.

In embodiments of the invention, the evaluation unit is designed to analytically solve the system of equations.

In embodiments of the invention, the evaluation unit is designed to numerically solve the system of equations.

In embodiments of the invention, the evaluation unit is designed to iteratively solve the system of equations.

In embodiments of the invention, the evaluation unit is designed to evaluate the measurement data acquired with the measurement devices using at least one AI system.

In embodiments of the invention, the evaluation unit is designed to evaluate the measurement data using a combination of analytical, numerical, iterative and/or AI-based methods.

In embodiments of the invention, the device for determining substance proportions of a fluid mixture of substances has at least one compensation device with which for example the temperature, the pressure, the volume flow and/or the humidity of the mixture of substances can be measured and can be used to compensate for at least one of the other measurements of the measurements carried out to determine the substance proportions.

In embodiments of the invention, the device for determining substance proportions of a fluid mixture of substances is designed for use in a medical technology application.

In embodiments of the invention, the device for determining substance proportions of a fluid mixture of substances is designed for determining at least one gas proportion of respiratory gas during ventilation of a patient.

A medical device according to the invention has at least one device according to the invention for determining substance proportions of a fluid mixture of substances.

In embodiments of the invention, the medical device is designed as a ventilator, wherein the mixture of substances is a gas mixture, namely respiratory gas.

In other embodiments of the invention, the medical device is designed for blood gas analysis.

In embodiments of the invention, the medical device is designed to use the determined substance proportions to adapt the control of a therapy module and/or an instruction module.

In embodiments of the invention, a therapy module is designed as the ventilation unit of a ventilator and, in embodiments of the invention, the instruction module is designed to output instructions to a human assistant.

A method for determining substance proportions of a fluid mixture of substances according to the invention can be used to determine at least one substance proportion of a fluid mixture of substances.

According to the invention, a method for determining substance proportions of a fluid mixture of substances comprises at least the following method steps:
Performing at least two different non-selective measurements, wherein one substance property of the mixture of substances is measured in each case and wherein the substance properties measured with the different measurements are independent of one another,
combining the measured substance properties to determine the proportion of at least one substance in the mixture of substances.

In embodiments of the method, at least X−1 different non-selective measuring methods or measuring devices which measure different, mutually independent substance properties of the mixture of substances are used for a number of X proportions of the fluid mixture of substances to be determined.

In embodiments of the method according to the invention, at least two of the non-selective measurements are performed simultaneously or at least partially overlapping in time.

In embodiments of the method according to the invention, all non-selective measurements are carried out simultaneously or at least partially overlapping in time.

In embodiments of the method according to the invention, the proportion of O2 and/or CO2 in the mixture of substances is determined.

In embodiments of the method according to the invention, at least one of the substance properties thermal conductivity, heat capacity, speed of sound (e.g. ultrasonic sensor), molar mass, dynamic viscosity, permittivity or refractive index or speed of light is measured.

In embodiments of the method according to the invention, at least two of the substance properties thermal conductivity, heat capacity, speed of sound, molar mass, dynamic viscosity, permittivity or refractive index or speed of light are measured.

In embodiments of the method according to the invention, in addition to the at least two non-selective measurements, at least one selective sensor is additionally used for the selective determination of at least one substance proportion in the mixture of substances.

In embodiments of the method according to the invention, a sensor for NDIR absorption measurement for determining the CO2 content, a lambda probe for determining the O2 content, a paramagnetic sensor for determining the O2 content, an optical sensor such as an optrode for measurement by means of fluorescence quenching (in particular a pO2 optrode), an electrochemical gas sensor or a humidity sensor (in particular by means of permittivity measurement) is used as a selective sensor.

In embodiments of the method according to the invention, it is known which substances the mixture of substances contains or which substances the mixture of substances contains that influence the properties of the mixture of substances to a relevant extent.

In embodiments of the method according to the invention, at least one substance property is determined iteratively. This allows the measurement accuracy to be improved in corresponding embodiments.

In embodiments of the method according to the invention, more than X−1 different non-selective measurement methods are used. This enables to increase the robustness and/or the accuracy of the measurement(s).

Furthermore, with the aid of at least one additional measuring method/measuring device, a plausibility check of the substance proportions determined from the other measurements and/or a check for unknown substances in the mixture of substances with a relevant influence on the measurement(s) is realized in embodiments of the invention.

In embodiments of the invention, at least one additional measuring method is used to measure a substance property slowly but accurately with a first measuring method and to detect rapid changes in the respective substance property with a second fast but possibly inaccurate measuring method in absolute terms, such as a strongly drifting measurement.

In embodiments of the invention, at least one measuring method is used which is influenced by at least two substance properties (e.g. thermal conductivity and heat capacity). The measuring method is used several times in different embodiments (for example, modulation of the temperature of the mixture of substances using a heater), with the influence of the different substance properties being weighted differently in each embodiment. In the example of thermal conductivity and heat capacity as substance properties, this would be, for example, the measurement of the temperature of the mixture of substances, whereby the temperature can be adjusted using a controllable heater. Depending on the speed of the modulation, the measurement is predominantly influenced by the thermal conductivity (slow modulation) or the heat capacity (fast modulation).

In embodiments of the invention, at least one measurement method is used in which the respective substance property is measured in two different states of the mixture of substances. For example, many substance properties are dependent on temperature and/or pressure. In corresponding embodiments of the invention, the mixture of substances is first measured at a first temperature T1 (e.g. ambient temperature) and/or a first pressure P1. Subsequently, the respective parameter is changed without changing the composition of the mixture of substances and the measurement is repeated. For example, the mixture of substances is heated (or cooled) to a temperature T2 or the pressure is changed to a value P2. If the substance properties of the individual components of the mixture of substances have significantly different temperature or pressure behavior, the new measurement provides new information about the composition of the mixture of substances.

In embodiments of the method according to the invention, the proportion of at least one substance in the mixture is determined by automatically setting up and solving a system of equations, which may be linear, from the measured substance properties.

In embodiments of the method according to the invention, the system of equations established with the aid of the determined substance properties is solved analytically.

In embodiments of the method according to the invention, the system of equations established using the determined substance properties is solved numerically.

In embodiments of the method according to the invention, the system of equations established using the determined substance properties is solved iteratively.

Such an iterative solution of the system of equations functions as follows in embodiments of the invention. Even if a substance property S1 cannot be used selectively to determine the proportion of a substance in the mixture of substances, it can be dominated by a substance A1. In the first iteration stage, it is assumed that the measurement of the substance property S1 is only determined by the substance A1. Then the proportion of substance A1 is first determined and the system of equations with X equations is reduced by 1.

In embodiments of the invention, at least one further substance property S2 which is independent of the substance property S1 is similarly used to estimate a substance proportion A2 or further substance proportions, so that the system of equations to be solved is further reduced.

In embodiments of the invention, at least one substance proportion is estimated in at least one iteration stage by an empirical value. In certain mixtures of substances, for example air or breathing air, certain substances (e.g. argon or nitrogen) normally occur in certain concentrations. In corresponding embodiments of the invention, these values are used at least for an initial estimation in the first iteration stage, so that the system of equations can be reduced accordingly.

In embodiments of the invention, substances that have a similar influence on a substance property are taken together during the corresponding iteration and treated as a single substance. In embodiments of the invention, substances which have a similar influence on all measured substance properties are considered globally as a single substance when solving the equation system, provided that a determination of the separate proportions of these substances in the mixture of substances is irrelevant in the respective application.

If, in the end, all substance proportions in the mixture of substances have been determined in the first iteration stage, these resulting values are used again in at least one next iteration stage, in which the influences of several or all substances on the respective substance properties are then taken into account, for improved determination of the substance proportions in the mixture of substances. In this way, the analysis is refined. The improvement in the accuracy of the determined substance proportions increases with further iteration stages.

In embodiments of the method according to the invention, the measurement data recorded by the measuring devices is evaluated using at least one AI system.

A combination of elements from the numerical, iterative and/or AI-supported determination of the substance proportions is also possible in embodiments of the invention.

In embodiments of the method according to the invention, at least one of the values temperature, pressure, volume flow and/or humidity of the mixture of substances is measured and used to compensate for at least one of the other measurements carried out to determine the substance proportions.

In embodiments of the method according to the invention, the method is used in a medical application.

In embodiments of the method according to the invention, at least one gas proportion of respiratory gas is determined during ventilation of a patient.

In embodiments of the method according to the invention, at least one device according to the invention for determining substance proportions of a fluid mixture of substances or a medical device according to the invention is used.

Exemplary embodiments of the invention are shown in the figures explained below. They show

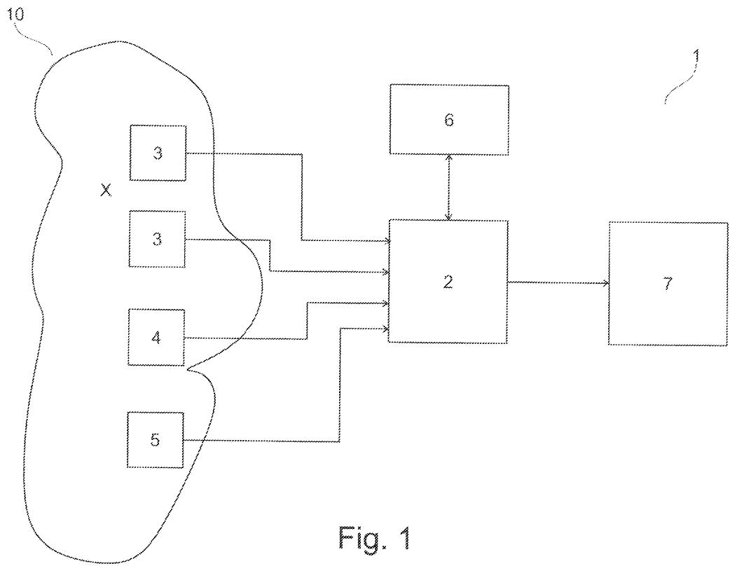

FIG. 1: A schematic block diagram of a device according to the invention for determining at least one substance proportion in a fluid mixture of substances,

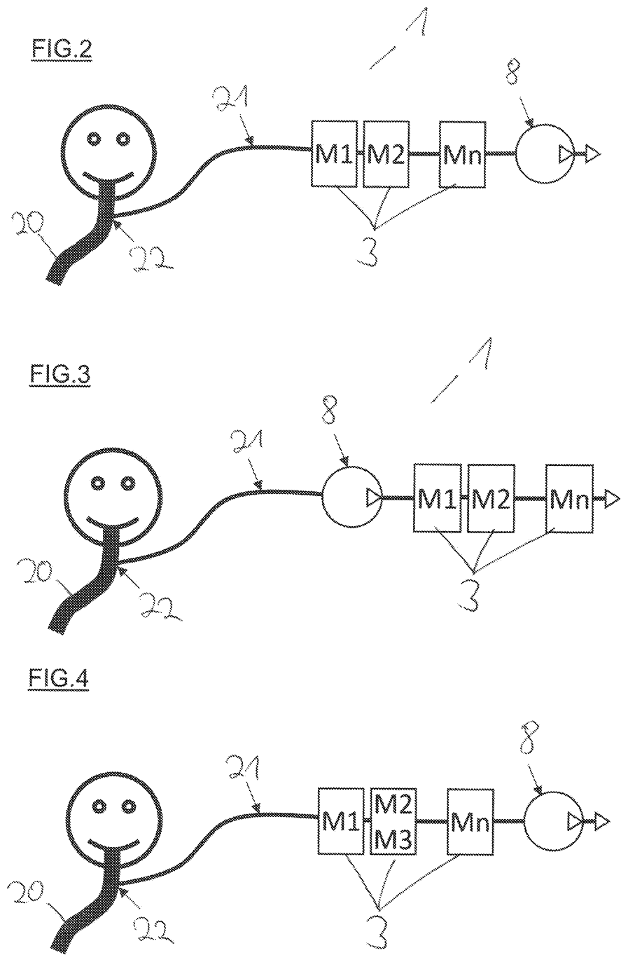

FIGS. 2 to 4: Schematic block diagrams of a device according to the invention for determining at least one substance proportion in a fluid mixture of substances in a side stream configuration in three different embodiments,

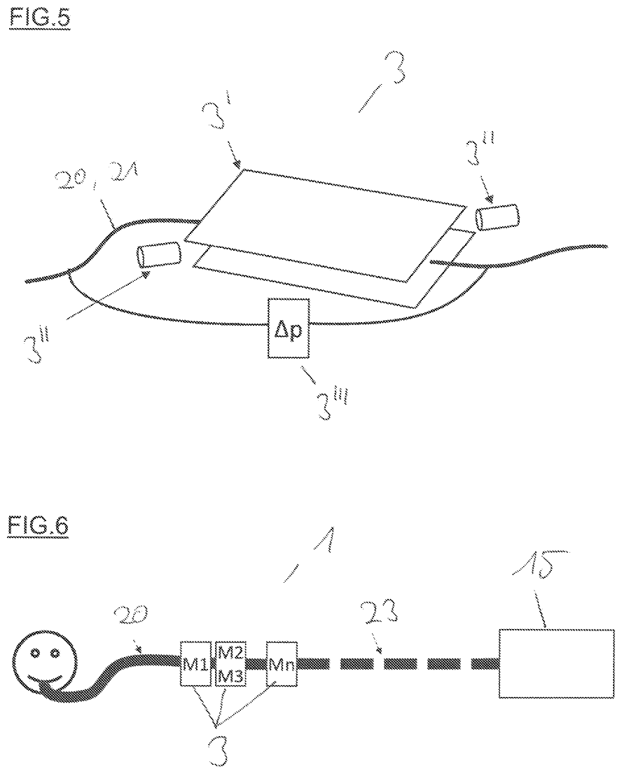

FIG. 5: A sensor configuration of a device according to the invention for the determination of at least one substance proportion in a fluid mixture of substances,

FIG. 6: A schematic block diagram of a device according to the invention for determining at least one substance proportion in a fluid mixture of substances in a main stream configuration,

FIG. 7: An illustration of various iteration stages in a corresponding method according to the invention for determining at least one substance proportion in a fluid mixture of substances, and

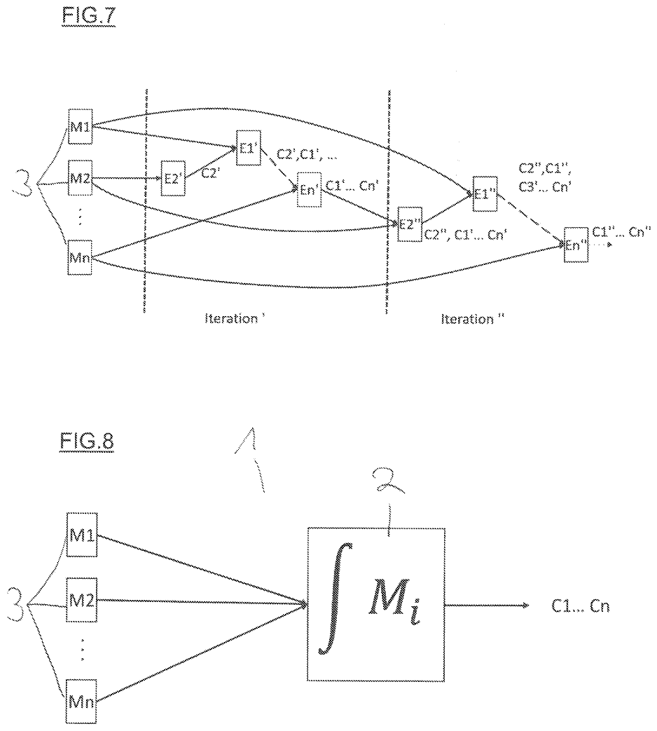

FIG. 8: A schematic illustration of the measured value processing according to the invention.

FIG. 1 shows a schematic block diagram of a device (1) for determining at least one substance proportion in a fluid mixture of substances (10) according to the invention. The device (1) for determining at least one substance proportion in a fluid mixture of substances (10) comprises an evaluation unit (2), two non-selective sensors (3), a selective sensor (4) and a compensation device (5) for capturing measured values in relation to a fluid mixture of substances (10). The evaluation unit (2) is designed to retrieve and evaluate the measured values for determining at least one substance proportion of the fluid mixture of substances (10).

In the embodiment of the invention shown, the evaluation unit (2) can make use of an A1 system (6) to determine the substance proportions of the fluid mixture of substances (10).

The at least one determined substance proportion of the fluid mixture of substances (10) can be output with the aid of an output unit (7), for example as an indication on a display, as an acoustic output or as a digital data packet.

FIGS. 2, 3 and 4 show various embodiments of a device (1) for determining at least one substance proportion of a fluid mixture of substances (10) according to the invention in off-line configurations.

FIG. 2 shows the arrangement of non-selective sensors (3) in a side stream (21), which branches off from the main stream (20) of the fluid mixture (10) at a tapping point (22). The fluid mixture of substances (10) is conveyed into the side stream (21) and past the sensors (3) for measurement by means of a suction pump (8) arranged downstream of the sensors (3) in the direction of flow. According to the invention, embodiments with at least one selective sensor are also possible.

According to the embodiment shown in FIG. 3, the suction pump (8) is positioned in the side stream (21) upstream of the sensors (3) in the direction of flow.

FIG. 4 shows an embodiment with two sensors (3) arranged in parallel, characterized by M2 and M3.

FIG. 5 shows a sensor configuration (3) for a parallel measurement of at least three measured values. The fluid mixture of substances (10) can be fed into the area of the sensor configuration (3) using a line, here designed as a main stream line (20) or side stream line (21). The sensor configuration (3) comprises a permittivity sensor (3′) designed as a plate capacitor, a speed of sound and/or volume flow sensor (3″) realized with the aid of an ultrasonic sensor and a viscosity sensor (3″) realized with the aid of a differential pressure sensor.

The differential pressure can be detected using pressure sensors arranged at the inlet and outlet of the plate capacitor. The space between the capacitor plates is also used for ultrasonic measurement.

Appropriate arrangements make it possible to realize compact sensor configurations (3), which are particularly important for mobile applications.

FIG. 6 shows a medical device (15) designed as a ventilator, which has a device (1) for determining at least one substance proportion in a fluid mixture of substances (10), which is integrated into the tube system (23) of the ventilator in relation to the sensors, shown here as non-selective sensors (3).

FIG. 7 shows the iterative determination of substance proportions of a fluid mixture of substances (10) according to advantageous embodiments of the invention. Based on the initially determined measured values M1, M2 . . . Mn, the substance proportions C1′, C2′, . . . Cn′ are determined in the first step, whereby the estimates E1′, E2′, . . . En′ are used. Based on the determined substance proportions, improved estimates E1″, E2″, . . . En″ are used in the second iteration stage to determine the now more precise substance proportions C1″, C2″, . . . Cn″.

Depending on the time and computing resources available, embodiments according to the invention with more than two iteration stages can also be realized.

FIG. 8 schematically shows the mathematical evaluation of the measured values recorded by the sensors (3) using the evaluation unit (2) to determine the substance proportions C1, . . . Cn of a fluid mixture of substances (10).

1-23. (canceled)

24. A device for determining substance proportions of a fluid mixture of substances, comprising: at least two different non-selective measuring devices which are each designed to measure a substance property of the mixture of substances, the substance properties measured with the measuring devices being different and independent of one another; at least one evaluation unit for evaluating measured values of the at least two different substance properties recorded by the measuring devices and for determining at least one substance proportion in the mixture of substances from a combination of the measured substance properties.

25. The device according to claim 24, wherein the at least two measuring devices include at least X−1 non-selective measuring devices for a number X of proportions of the fluid mixture of substances to be determined, with which different, mutually independent substance properties of the mixture of substances are measureable.

26. The device according to claim 24, wherein at least two of the non-selective measuring devices are configured to carry out the respective measurements simultaneously or at least partially overlapping in time or have a measurable or known time offset, so that the individual measurements can be synchronized.

27. The device according to claim 24, wherein the device is configured to determine a proportion of O2 and/or CO2 in the mixture of substances.

28. The device according to claim 24, wherein at least one of the measuring devices has at least one sensor for determining one of the substance properties thermal conductivity, heat capacity, speed of sound, molar mass, dynamic viscosity, permittivity or refractive index or speed of light.

29. The device according to claim 24, further comprising at least one selective sensor for selective determination of at least one substance proportion in the mixture of substances.

30. The device according to claim 24, wherein the device is configured for iterative determination of at least one substance property.

31. The device according to claim 24, further comprising an evaluation unit configured to determine a proportion of at least one substance in the mixture of substances with aid of setting up a system of equations from the measured substance properties and the solution of the system of equations.

32. The device according to claim 24, further comprising a compensation device configured to measure temperature, pressure, volume flow and/or humidity of the mixture of substances and compensate for at least one of the other measurements carried out to determine the substance proportions.

33. The device according to claim 24, wherein the device is configured to determine at least one gas proportion of respiratory gas during ventilation of a patient.

34. A medical device, comprising: at least one device for determining substance proportions of a fluid mixture of substances according to claim 24.

35. The medical device according to claim 34, wherein the device is a ventilator and the mixture of substances is a gas mixture.

36. A method for determining substance proportions of a fluid mixture of substances, comprising the steps of:
carrying out at least two different non-selective measurements, wherein in each case one substance property of the mixture of substances is measured and wherein the substance properties measured with the different measurements are independent of one another; and
combining the measured substance properties to determine the proportion of at least one substance in the mixture of substances.

37. The method according to claim 36, including using at least X−1 different non-selective measuring methods or measuring devices which measure different, mutually independent substance properties of the mixture of substances for a number of X proportions of the fluid mixture of substances to be determined.

38. The method according to claim 37, including carrying out at least two of the non-selective measurements simultaneously or at least partially overlapping in time or with a measurable or known time offset, the individual measurements being synchronized.

39. The method according to claim 36, including determining a proportion of O2 and/or CO2 in the mixture of substances.

40. The method according to claim 36, including measuring at least one of the substance properties thermal conductivity, heat capacity, speed of sound, molar mass, dynamic viscosity, permittivity or refractive index or speed of light.

41. The method according to claim 36, wherein, in addition to the at least two non-selective measurements, at least one selective sensor is additionally used for the selective determination of at least one substance proportion in the mixture of substances.

42. The method according to claim 36, including iteratively determining at least one substance property.

43. The method according to claim 36, wherein the proportion of at least one substance in the mixture of substances is determined by automatically setting up and solving a system of equations from the measured substance properties.

44. The method according to claim 36, including measuring at least one value of temperature, pressure, volume flow and/or humidity of the mixture of substances and using the at least one value to compensate for at least one of the other measurements carried out to determine the substance proportions.

45. The method according to claim 36, including determining at least one gas proportion of respiratory gas during ventilation of a patient.

46. The method according to claim 36, including using at least one device for determining substance proportions of a fluid mixture of substances, the at least one device including: at least two different non-selective measuring devices which are each designed to measure a substance property of the mixture of substances, the substance properties measured with the measuring devices being different and independent of one another; at least one evaluation unit for evaluating measured values of the at least two different substance properties recorded by the measuring devices and for determining at least one substance proportion in the mixture of substances from a combination of the measured substance properties.
